(19) United States
(12) Patent Application Publication
(10) Pub. No.: US 2025/0367029 A1
(43) Pub. Date: Dec. 4, 2025

(54) ANTERIOR CHAMBER MAINTAINER (ACM)

(71) Applicant: JEFFREY WHITSETT, Houston, TX (US)

(72) Inventor: JEFFREY WHITSETT, Houston, TX (US)

(21) Appl. No.: 18/731,293

(22) Filed: Jun. 2, 2024

(51) Int. Cl. A61F 9/007; A61M 37/00

(52) U.S. Cl. CPC A61F 9/007; A61M 37/00; A61M 2202/0007; A61M 2202/04; A61M 2210/0612

(57) ABSTRACT

An anterior chamber maintainer (ACM), which is to be utilized in connection with maintaining physiological intraocular pressure during intraocular surgery, is disclosed and comprises an axially elongated tubular member which is adapted to be fluidically connected at a first end to a supply of balanced salt solution (BSS) for introduction into the anterior chamber of an eye so as to maintain adequate intraocular pressure while performing any intraocular surgery. A second opposite end of the anterior chamber maintainer (ACM) is provided with a sharply pointed end for insertion through a paracentesis incision formed within the limbus region of the eye, and a non-deformable, incompressible, tapered, annular bulbous portion is interposed between the first and second ends such that as the second end passes through the paracentesis incision, the non-deformable, incompressible, tapered, annular bulbous portion will cause the paracentesis incision to deform or expand radially outwardly, and after passing through the paracentesis incision, the non-deformable, incompressible, annular bulbous portion will trap the anterior chamber maintainer (ACM) within the anterior chamber of the eye.

### FIELD OF THE INVENTION

The present invention relates generally to medical equipment, and more particularly to a new and improved anterior chamber maintainer (ACM) which is to be utilized in connection with maintaining physiological intraocular pressure during intraocular surgery. Still more particularly, the new and improved anterior chamber maintainer (ACM) effectively comprises a device which enables the cataract surgeon to infuse balanced salt solution (BSS) into the anterior chamber of the eye so as to maintain adequate intraocular pressure while performing any intraocular surgery.

### BACKGROUND OF THE INVENTION

Conventionally, anterior chamber intraocular chamber maintainers (ACMs) have been used during different types of intraocular surgeries all of which require that the intraocular pressure be maintained within the anterior segment of the eye while performing such intraocular surgery. Such intraocular surgery may comprise, for example, cataract surgery wherein lens material is removed from the eye, or vitrectomy surgery wherein vitreous material such as, for example, floaters, are severed and removed from the posterior segment of the eye. Such conventional anterior chamber intraocular pressure maintainers (ACMs) utilize balanced salt solution (BSS) infused into anterior chamber of the eye. The constant infusion of balanced salt solution (BSS), and the maintenance of stable intraocular pressure within the eye, are both necessary in order to achieve proper fluidics and function during all intraocular surgeries. During cataract surgery, which is the most common ophthalmic surgical procedure performed within this country, balanced salt solution (BSS) is infused in a coaxial manner to inflate the eye while simultaneously removing the natural crystalline lens. During vitrectomy surgery, however, an infusion cannula is inserted through the pars plana of the eye so as to maintain the intraocular pressure while the vitreous material is being severed and removed. The problem with such an infusion cannula, however, resides in the fact that in order to render such to be fixed or stable relative to the eye, the infusion cannula is larger and often requires sutures to be closed adequately. Therefore, not only is it necessary for the infusion cannula to be entered through the sclera in the region of the pars plana, but then after the vitrectomy has been completed, the sutures will be required to close the scleral opening. All of these procedures not only require additional time to be invested by means of the vitrectomy surgeon and his or her staff, but such procedures may increase the morbidity rate as well as being more adversely impactful upon the patient's recovery.

A need therefore exists for a new and improved anterior chamber maintainer (ACM). Another need exists in the art for a new and improved anterior chamber maintainer (ACM) which can be used during different types of intraocular surgeries all of which require that the intraocular pressure be maintained within the anterior segment of the eye while performing such intraocular surgery. Still another need exists in the art for a new and improved anterior chamber maintainer (ACM) which can be used during different types of intraocular surgeries all of which require that the intraocular pressure be maintained within the anterior segment of the eye while performing such intraocular surgery and which is easy to manipulate by the surgeon. Yet another need exists in the art for a new and improved anterior chamber maintainer (ACM) which can be used during different types of intraocular surgeries all of which require that the intraocular pressure be maintained within the anterior segment of the eye while performing such intraocular surgery, which is easy to manipulate by the surgeon, and which comprises an infusion cannula to supply balanced salt solution (BSS) into the anterior chamber of the eye in view of the fact that the constant infusion of balanced salt solution (BSS), and the maintenance of stable intraocular pressure within the eye, are both necessary in order to achieve proper fluidics and function during all intraocular surgeries.

A further need exists in the art for a new and improved anterior chamber maintainer (ACM) which can be used during different types of intraocular surgeries all of which require that the intraocular pressure be maintained within the anterior segment of the eye while performing such intraocular surgery, which is easy to manipulate by the surgeon, which comprises an infusion cannula to supply balanced salt solution (BSS) into the anterior chamber of the eye in view of the fact that the constant infusion of balanced salt solution (BSS), and the maintenance of stable intraocular pressure within the eye, are both necessary in order to achieve proper fluidics and function during all intraocular surgeries, and which is capable of effectively being fixed to or disposed in a stable state relative to the eye without the need for suturing the infusion cannula to any portion of the eye. A still further need exists in the art for a new and improved anterior chamber maintainer (ACM) which can be used during different types of intraocular surgeries all of which require that the intraocular pressure be maintained within the anterior segment of the eye while performing such intraocular surgery, which is easy to manipulate by the surgeon, which comprises an infusion cannula to supply balanced salt solution (BSS) into the anterior chamber of the eye in view of the fact that the constant infusion of balanced salt solution (BSS), and the maintenance of stable intraocular pressure within the eye, are both necessary in order to achieve proper fluidics and function during all intraocular surgeries, and which is capable of effectively being fixed to or disposed in a stable state relative to the eye without the need for suturing the infusion cannula to any portion of the eye such that there are no sutures to be placed or removed by the surgeon or any member of his or her staff which will lead to a more positive surgical result for the patient with respect to morbidity and recovery.

### OVERALL OBJECTIVES OF THE PRESENT INVENTION

An overall objective of the present invention is to provide a new and improved anterior chamber maintainer (ACM). Another overall objective of the present invention is to provide a new and improved anterior chamber maintainer (ACM) which can be used during different types of intraocular surgeries all of which require that the intraocular pressure be maintained within the anterior chamber of the eye while performing such intraocular surgery. Still another overall objective of the present invention is to provide a new and improved anterior chamber maintainer (ACM) which can be used during different types of intraocular surgeries all of which require that the intraocular pressure be maintained within the anterior segment of the eye while performing such intraocular surgery and which is easy to manipulate by the surgeon. Yet another overall objective of the present invention is to provide a new and improved anterior chamber maintainer (ACM) which can be used during different types of intraocular surgeries all of which require that the intraocular pressure be maintained within the anterior segment of the eye while performing such intraocular surgery, which is easy to manipulate by the surgeon, and which comprises an infusion cannula to supply balanced salt solution (BSS) into the anterior chamber of the eye in view of the fact that the constant infusion of balanced salt solution (BSS), and the maintenance of stable intraocular pressure within the eye, are both necessary in order to achieve proper fluidics and function during all intraocular surgeries.

A further overall objective of the present invention is to provide a new and improved anterior chamber maintainer (ACM) which can be used during different types of intraocular surgeries all of which require that the intraocular pressure be maintained within the anterior segment of the eye while performing such intraocular surgery, which is easy to manipulate by the surgeon, which comprises an infusion cannula to supply balanced salt solution (BSS) into the anterior chamber of the eye in view of the fact that the constant infusion of balanced salt solution (BSS), and the maintenance of stable intraocular pressure within the eye, are both necessary in order to achieve proper fluidics and function during all intraocular surgeries, and which is capable of effectively being fixed to or disposed in a stable state relative to the eye without the need for suturing the infusion cannula to any portion of the eye. A still further overall objective of the present invention is to provide a new and improved anterior chamber maintainer (ACM) which can be used during different types of intraocular surgeries all of which require that the intraocular pressure be maintained within the anterior chamber of the eye while performing such intraocular surgery, which is easy to manipulate by the surgeon, which comprises an infusion cannula to supply balanced salt solution (BSS) into the anterior chamber of the eye in view of the fact that the constant infusion of balanced salt solution (BSS), and the maintenance of stable intraocular pressure within the eye, are both necessary in order to achieve proper fluidics and function during all intraocular surgeries, and which is capable of effectively being fixed to or disposed in a stable state relative to the eye without the need for suturing the infusion cannula to any portion of the eye such that there are no sutures to be removed by the surgeon or any member of his or her staff which will lead to a more positive surgical result for the patient with respect to morbidity and recovery.

### SUMMARY OF THE INVENTION

The foregoing and other objectives are achieved in accordance with the principles and teachings of the present invention through means of an anterior chamber maintainer (ACM) which comprises a stacked link structure or relatively large diameter tubular member which not only defines a fluid conduit for receiving an externally disposed sleeve member or infusion line, which may be fabricated from any conventional medical grade irrigation tubing and which is fixedly connected to a continuous supply of balanced salt solution (BSS) to be introduced into the anterior chamber of the eye, but in addition, the stacked link structure or relatively large diameter tubular member effectively defines a handle which may be grasped by the surgeon to easily manipulate the anterior chamber maintainer (ACM) if desired or necessary. A one millimeter (1 mm) paracentesis incision is made at the limbus of the eye, and it is seen that the stacked link structure or relatively large diameter tubular member is integrally mounted upon a base member or platform which is adapted to be engaged with an external portion of the eye when a distal end portion of a relatively small diameter tubular member, integrally connected to the base member or platform and provided with a sharply pointed end, is inserted through the paracentesis incision. In addition, an axially central portion of the relatively small diameter tubular member, that is located between the base member and the sharpened or pointed end, is provided with a non-deformable, incompressible, annular, rounded, bulbous portion or bulbar. The tubular member is fabricated from a suitable medical grade material, such as, for example, a 21-23 g material, comprising stainless steel or titanium, which is similar to that utilized in existing cannulas and which exhibits sufficient axial stiffness to permit the tubular member to pass through the paracentesis incision.

More particularly, as the distal end of the relatively small diameter tubular member passes through the paracentesis, the non-deformable, incompressible annular, rounded or bulbous portion effectively force the paracentesis incision to expand radially outwardly so as to permit the non-deformable, annular, rounded or bulbous portion to pass through the paracentesis incision, and then, as soon as the non-deformable, incompressible, annular, rounded or bulbous portion has effectively passed through the paracentesis incision, the paracentesis incision will effectively regain its original, non-expanded configuration or size. In this manner, it is further appreciated that as a result of the non-deformable, incompressible annular, rounded or bulbous passing through the paracentesis incision, the bulbous portion will now effectively stabilize the anterior chamber maintainer (ACM) within the anterior chamber of the eye such that the infusion line or sleeve member is now effectively connected to the eye whereby the balanced salt solution (BSS) can be fluidically conducted into the anterior chamber of the eye. This connection of the infusion line to the eye, by means of the new and improved anterior chamber maintainer (ACM) also permits the surgeon to grasp the anterior chamber maintainer (ACM) by means of the stacked link structure and the relatively large diameter tubular member so as to effectively rotate, pivot, or otherwise directionally orient or manipulate the eye so as to aid the surgeon's visualization during performance of the surgery without any fear that the infusion line will become disconnected from the eye. The surgeon may also leave the anterior chamber maintainer (ACM) in position by itself so as to allow the surgeon to utilize both hands while performing the intraocular surgery. This hands-free mode of operation is particularly important during the performance of complex anterior segment and lens exchange procedures by the ophthalmologist/surgeon. When the surgery is complete, the anterior chamber maintainer (ACM) can be readily retracted out from the eye by simply rotating and pulling on the infusion line/anterior chamber maintainer (ACM) assembly whereby the non-deformable, annular, round or bulbous portion of the anterior chamber maintainer (ACM) will again radially deform the paracentesis incision so as to permit the anterior chamber maintainer (ACM) to pass back outwardly through the paracentesis incision.

### BRIEF DESCRIPTION OF THE DRAWINGS

Various other features and attendant advantages of the present invention will be more fully appreciated from the following detailed description when considered in connection with the accompanying drawing wherein:

FIG. 1 is a schematic cross-sectional view of a human eye schematically illustrating the insertion of the new and improved anterior chamber maintainer (ACM) when inserted into the eye;

FIG. 2 is an enlarged, schematic front elevational view of the new and improved anterior chamber maintainer (ACM) clearly illustrating the structure of the anterior chamber maintainer (ACM) as comprising the stacked link structure or relatively large diameter tubular member which comprises a plurality of serially arranged frusto-conical portions which define lower end annular stepped portions over which the infusion line is disposed and tightly secured, the sharply pointed distal end of the anterior chamber maintainer (ACM) which is adapted to be inserted through the paracentesis incision, and the non-deformable, incompressible, annular, rounded or bulbous portion of the anterior chamber maintainer (ACM) which passes through the paracentesis incision and maintains the anterior chamber maintainer (ACM) within the eye;

FIG. 3 is an enlarged, schematic cross-sectional view of the new and improved anterior chamber maintainer (ACM) as taken along the lines 3-3 of FIG. 2, clearly showing the sharply pointed distal end portion of the new and improved anterior chamber maintainer (ACM) which is adapted to easily penetrate the paracentesis incision formed within the limbus portion of the eye; and

FIG. 4 is an enlarged, schematic view of the encircled region 4 as shown in FIG. 3 so as to more clearly show the lower distal end portion of the new and improved anterior chamber maintainer (ACM) which includes the sharply pointed end portion and the non-deformable, incompressible annular, round or bulbous portion of the new and improved anterior chamber maintainer (ACM).

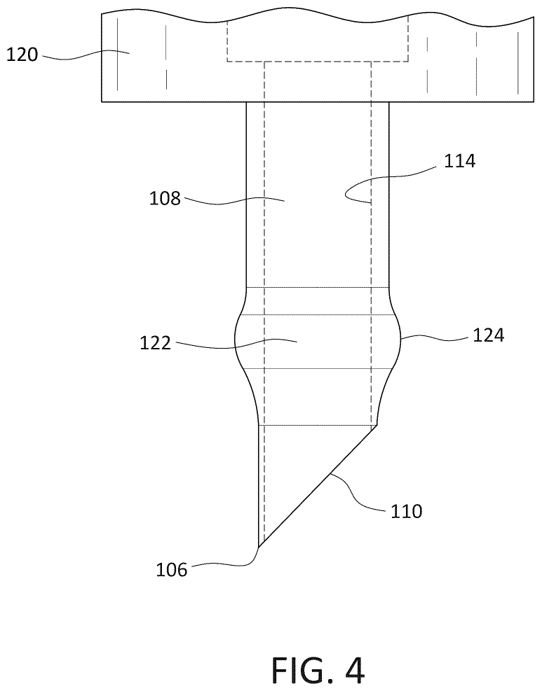

### DETAILED DESCRIPTION OF THE PRESENT INVENTION

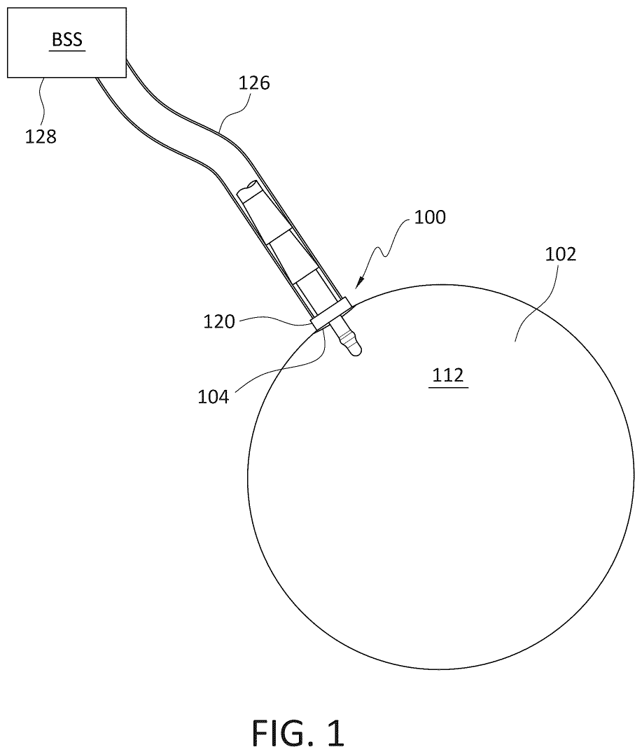

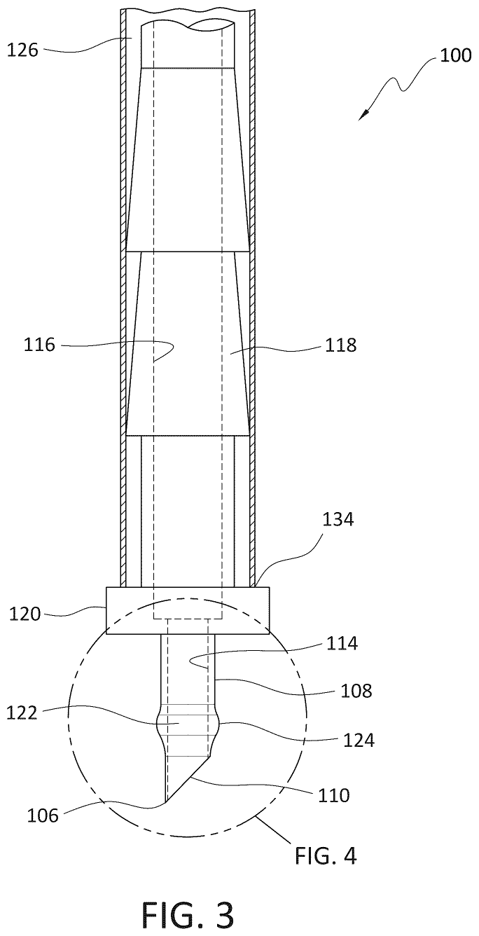

With reference being made to FIGS. 1,3 and 4, the new and improved anterior chamber maintainer (ACM) of the present invention is disclosed, is generally indicated by the reference character 100, and is schematically illustrated as it would be secured to and within a person's eye 102 during, for example, vitrectomy surgery. More particularly, a paracentesis incision 104 of approximately one millimeter (1 mm) is created at the limbus section of the eye 102, which is defined as the junction of the cornea and sclera of the eye, and a sharply pointed distal end portion 106 of a relatively small diameter tubular member 108 of the anterior chamber maintainer (ACM) 100, as can best be appreciated from FIG. 3, is adapted to be inserted through the paracentesis incision 104 such that an open end portion 110 of the sharply pointed distal end portion 106 of the anterior chamber maintainer (ACM) 100 is disposed within the anterior chamber 112 of the eyeball 102. Still further, it is seen that the relatively small diameter tubular member 108 defines an internal tubular fluid conduit 114 which is in fluidic communication with a larger, internal tubular fluid conduit 116 which is defined within a relatively large diameter tubular member 118. The relatively small diameter tubular member 108 and the relatively large diameter tubular member 118 are integrally connected together by means of a transversely oriented base member or platform 120 which, as can be appreciated from FIG. 1, is adapted to be seated upon an exterior surface portion of the eye 102 once the sharply pointed distal end portion 106 of the anterior chamber maintainer (ACM) 100 has been inserted through the paracentesis incision 104. It is also seen that an axially central portion 122 of the relatively small tubular member 108 comprises a non-deformable, incompressible, tapered, annular, round, or bulbous portion 124.

The relatively small diameter tubular member 108 is fabricated from a suitable medical grade material, such as, for example, 21-23 g material, comprising stainless steel or titanium, which is similar to that utilized to manufacture cannulas for fluid delivery and which exhibits sufficient axial stiffness to permit the relatively small diameter tubular member 108 to pass through the paracentesis incision 104. In a similar manner, the non-deformable, incompressible tapered, annular, round or bulbous portion 124 is fabricated from a suitable material, such as, for example, stainless steel, titanium, or the like, so as to effectively cause the paracentesis incision to be deformed radially outwardly and thereby permit the non-deformable, incompressible tapered, annular, round or bulbous portion 124 to pass through the paracentesis incision 104, whereupon having passed through the paracentesis incision 104, the paracentesis incision 104 can regain its original non-deformed position or state. More particularly, as the relatively small diameter tubular member 106 passes through the paracentesis incision 104, the non-deformable, incompressible tapered, annular, round or bulbous portion 124 will effectively force the paracentesis incision 104 to expand radially outwardly so as to permit the axially central portion 122 of the relatively small diameter tubular member 108 to pass through the paracentesis incision 104, however, as soon as the non-deformable, incompressible tapered, annular, round or bulbous member or portion 124 has effectively passed through the paracentesis incision 104, the paracentesis incision 104 will regain its original, non-deformed state configuration. In this manner, it is further appreciated that as a result of the bulbous portion 124 having passed through the paracentesis incision 104, the bulbous portion 124 will now effectively trap the anterior chamber maintainer (ACM) 100 within the anterior chamber 112 of the eye 102 such that an infusion line 126, as can best be seen in FIGS. 1 and 3, can be effectively connected to the relatively large diameter tubular member 118 of the anterior chamber maintainer (ACM) 100 whereby a balanced salt solution (BSS) can be fluidically conducted into the anterior chamber 112 of the eye 102 from a suitable source of balanced salt solution (BSS) 128.

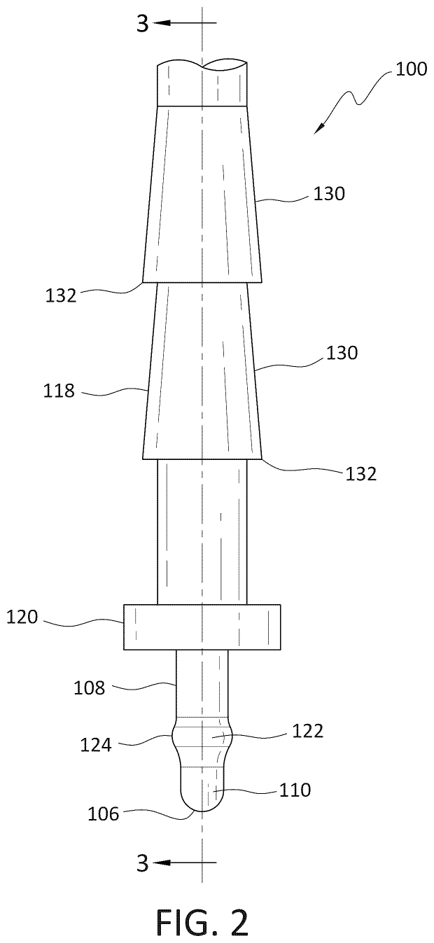

Continuing further, and with particular reference being made to FIGS. 2 and 3, it is seen that the relatively large diameter tubular section 118 of the anterior chamber maintainer (ACM) 100 comprises a plurality of frusto-conical portions or segments 130 integrally connected together and which define lower end annular stepped portions 132 over which the infusion line 126 is disposed and tightly secured in view of the fact that the infusion line 126 will conform to the external geometries of the frusto-conical portions or segments 130 whereby the annular stepped portions 132 will effectively prevent the infusion line 126 from becoming disconnected from the relatively large diameter tubular section 118 of the anterior chamber maintainer (ACM) 100. As a result of the aforenoted structure, the surgeon, performing a particular intraocular surgery, is able to easily grasp the anterior chamber maintainer (ACM) 100 by means of the relatively large diameter tubular member 118, and the infusion line 126 fluidically connected thereto, so as to effectively rotate, pivot, or otherwise directionally orient or manipulate the anterior chamber maintainer (ACM) 100 and the eye 102 in any particular direction and within any plane. These movements aid the visualization of the surgeon while he or she is performing the intraocular surgery without any fear that the infusion line 126 will become disconnected from the eye 102. It is also noted that as a result of the unique structure that comprises the anterior chamber maintainer (ACM) 100, the anterior chamber maintainer (ACM) 100 can be readily retracted out from the eye 102 by simply rotating and pulling on the infusion line/anterior chamber maintainer (ACM) assembly 126/100 whereby the axially central bulbous portion 124 of the anterior chamber maintainer (ACM) 100 will again cause the paracentesis incision 104 to be deformed or expanded radially outwardly so as to permit the anterior chamber maintainer (ACM) 100 to pass back outwardly through the paracentesis incision 104. It is lastly noted that when the infusion line 126 is mounted upon the relatively large diameter tubular section 118 of the anterior chamber maintainer (ACM) 100, the open end portion of the infusion line 126 will be seated upon the upper surface portions of the transversely oriented base member or platform 120 as at 134, as is shown in FIG. 3.

Obviously, many variations and modifications of the present invention are possible in light of the above teachings. It is therefore to be understood that within the scope of the appended claims, the present invention may be practiced otherwise than as specifically described herein.

What is claimed and desired to be protected by Letters Patent, is:

1. A new and improved anterior chamber maintainer (ACM) for fixation within an eye during intraocular surgery, comprising:
an axially elongated tubular member comprising a first open end portion which is adapted to be fluidically connected to a balanced salt solution (BSS) infusion line for receiving a supply of balanced salt solution (BSS) to be introduced into an anterior chamber of an eye during intraocular surgery;
said axially elongated tubular member comprising a second open end portion, opposite said first end portion, adapted to be disposed within the anterior chamber of the eye such that the balanced salt solution (BSS) from said balanced salt solution (BSS) infusion line can enter the anterior chamber of the eye so as to provide and maintain adequate pressure within the anterior chamber of the eye during intraocular surgery; and
a tapered, annular, non-deformable, incompressible, round or bulbous portion which is mounted upon said axially elongated tubular member so as to be interposed between said first and second open end portions of said axially elongated tubular member and which is adapted to cause a paracentesis incision, through which said second open end portion of said axially elongated tubular member has passed when inserted into the anterior chamber of the eye, to be radially expanded after said second open end portion of said axially elongated tubular member has passed through the paracentesis incision made within the eye and which will permit the paracentesis incision to regain its original configuration once said non-deformable, incompressible annular, round or bulbous portion of said axially elongated tubular member has passed through the paracentesis incision such that said non-deformable, incompressible tapered, annular, round or bulbous portion of said axially elongated tubular member effectively traps said anterior chamber maintainer (ACM) within the eye such that balanced salt solution (BSS) can be continuously supplied to the anterior chamber of the eye without fear that said balanced salt solution (BSS) infusion line will be fluidically disconnected from the anterior chamber of the eye.

2. The anterior chamber maintainer (ACM) as set forth in claim 1, wherein:
said second open end portion of said axially elongated tubular member comprises a sharply pointed distal end portion for facilitating insertion of said second open end portion and said non-deformable, incompressible annular, round or bulbous portion of said axially elongated tubular member through the paracentesis incision formed within the eye.

3. The anterior chamber maintainer (ACM) as set forth in claim 1, wherein:
said first open end portion of said axially elongated tubular member is defined upon a first relatively large diameter tubular portion of said axially elongated tubular member; and
said second open end portion of said axially elongated tubular member is defined upon a second relatively small diameter tubular portion of said axially elongated tubular member.

4. The anterior chamber maintainer (ACM) as set forth in claim 3, wherein:
said first relatively large diameter tubular portion of said axially elongated tubular member comprises a plurality of frusto-conical portions or segments having annular stepped portions which engage said balanced salt solution (BSS) infusion line such that said balanced salt solution (BSS) infusion line is not easily separated from said first relatively large diameter tubular portion of said axially elongated tubular member such that fluid flow of said balanced salt solution (BSS) from said infusion line into the anterior chamber of the eye is ensured.

5. The anterior chamber maintainer (ACM) as set forth in claim 3, wherein:
said first relatively large diameter tubular portion of said axially elongated tubular member effectively defines a handle by means of which the surgeon may manipulate said anterior chamber maintainer (ACM) in any direction, within any plane, once said non-deformable, incompressible tapered, annular, round or bulbous portion has passed through the paracentesis incision and is disposed within the anterior chamber of the eye.

6. The anterior chamber maintainer (ACM) as set forth in claim 1, wherein:
said axially elongated tubular member is fabricated from 21-23 g medical grade equipment material.

7. The anterior chamber maintainer (ACM) as set forth in claim 1, wherein:
said infusion line is fabricated from medical grade irrigation tubing.

8. The anterior chamber maintainer (ACM) as set forth in claim 1, wherein:
said non-deformable, incompressible tapered, annular, round or bulbous portion is fabricated from a suitable material selected from the group comprising stainless steel and titanium.

9. The anterior chamber maintainer (ACM) as set forth in claim 1, wherein:
said disposition of said anterior chamber maintainer (ACM) within said anterior chamber of the eye permits said anterior chamber maintainer (ACM) to be self-maintaining so as to permit the surgeon to utilize both hands during intraocular surgery.
